SEQUENCE LISTING (24 sequences)

SEQ ID NO: 1 - DNA, 2181 bp, Homo sapiens
ggcacgagggatgcaaggagatgagacagttagatttacttcctcttttctaatctgagaggtttcatgttgaagaaaatcagtgttggggttgcaggagacctaaacacagtcaccatgaagctgggctgtgtcctcatggcctgggccctctacctttcccttggtgtgctctgggtggcccagatgctactggctgccagttttgagacgctgcagtgtgagggacctgtctgcactgaggagagcagctgccacacggaggatgacttgactgatgcaagggaagctggcttccaggtcaaggcctacactttcagtgaacccttccacctgattgtgtcctatgactggctgatcctccaaggtccagccaagccagtttttgaaggggacctgctggttctgcgctgccaggcctggcaagactggccactgactcaggtgaccttctaccgagatggctcagctctgggtccccccgggcctaacagggaattctccatcaccgtggtacaaaaggcagacagcgggcactaccactgcagtggcatcttccagagccctggtcctgggatcccagaaacagcatctgttgtggctatcacagtccaagaactgtttccagcgccaattctcagagctgtaccctcagctgaaccccaagcaggaagccccatgaccctgagttgtcagacaaagttgcccctgcagaggtcagctgcccgcctcctcttctccttctacaaggatggaaggatagtgcaaagcagggggctctcctcagaattccagatccccacagcttcagaagatcactccgggtcatactggtgtgaggcagccactgaggacaaccaagtttggaaacagagcccccagctagagatcagagtgcagggtgcttccagctctgctgcacctcccacattgaatccagctcctcagaaatcagctgctccaggaactgctcctgaggaggcccctgggcctctgcctccgccgccaaccccatcttctgaggatccaggcttttcttctcctctggggatgccagatcctcatctgtatcaccagatgggccttcttctcaaacacatgcaggatgtgagagtcctcctcggtcacctgctcatggagttgagggaattatctggccaccagaagcctgggaccacaaaggctactgctgaatagaagtaaacagttcatccatgatctcacttaaccaccccaataaatctgattctttattttctcttcctgtcctgcacatatgcataagtacttttacaagttgtcccagtgttttgttagaataatgtagttaggtgagtgtaaataaatttatataaagtgagaattagagtttagctataattgtgtattctctcttaacacaacagaattctgctgtctagatcaggaatttctatctgttatatcgaccagaatgttgtgatttaaagagaactaatggaagtggattgaatacagcagtctcaactgggggcaattttgccccccagaggacattgggcaatgtttggagacattttggtcattatacttggggggttgggggatggtgggatgtgtgtgctactggcatccagtaaatagaagccaggggtgccgctaaacatcctataatgcacagggcagtaccccacaacgaaaaataatctggcccaaaatgtcagttgtactgagtttgagaaaccccagcctaatgaaaccctaggtgttgggctctggaatgggactttgtcccttctaattattatctctttccagcctcattcagctattcttactgacataccagtctttagctggtgctatggtctgttctttagttctagtttgtatcccctcaaaagccattatgttgaaatcctaatccccaaggtgatggcattaagaagtgggcctttgggaagtgattagatcaggagtgcagagccctcatgattaggattagtgcccttatttaaaaaggccccagagagctaactcacccttccaccatatgaggacgtggcaagaagatgacatgtatgagaaccaaaaaacagctgtcgccaaacaccgactctgtcgttgccttgatcttgaacttccagcctccagaactatgagaaataaaattctgttgtttgtaaaaaaaaaaaaaaaaaa

SEQ ID NO: 2 - protein, 359 aa, Homo sapiens
MKLGCVLMAWALYLSLGVLWVAQMLLAASFETLQCEGPVCTEESSCHTEDDLTDAREAGFQVKAYTFSEPFHLIVSYDWLILQGPAKPVFEGDLLVLRCQAWQDWPLTQVTFYRDGSALGPPGPNREFSITVVQKADSGHYHCSGIFQSPGPGIPETASVVAITVQELFPAPILRAVPSAEPQAGSPMTLSCQTKLPLQRSAARLLFSFYKDGRIVQSRGLSSEFQIPTASEDHSGSYWCEAATEDNQVWKQSPQLEIRVQGASSSAAPPTLNPAPQKSAAPGTAPEEAPGPLPPPPTPSSEDPGFSSPLGMPDPHLYHQMGLLLKHMQDVRVLLGHLLMELRELSGHQKPGTTKATAE

SEQ ID NO: 3 - DNA, 21 bp, Artificial
  Primer sequence for BLSA
cagagcccccagctagagatc

SEQ ID NO: 4 - DNA, 19 bp, Artificial
  Primer sequence from BLSA
gtgcagcagagctggaagc

SEQ ID NO: 5 - DNA, 20 bp, Artificial
  Primer sequence for BLSA
gcagtggcatcttccagagc

SEQ ID NO: 6 - DNA, 21 bp, Artificial
  Primer sequence for BLSA
cagatgctgtttctgggatcc

SEQ ID NO: 7 - DNA, 21 bp, Artificial
  Primer sequence for BLSA
gatcagagtgcagggtgcttc

SEQ ID NO: 8 - DNA, 20 bp, Artificial
  Primer sequence for BLSA
ggattcaatgtgggaggtgc

SEQ ID NO: 9 - DNA, 21 bp, Artificial
  Primer sequence for BLSA
gtgagggacctgtctgcactg

SEQ ID NO: 10 - DNA, 19 bp, Artificial
  pRIMER sequence for BLSA
agtcatcctccgtgtggca

SEQ ID NO: 11 - DNA, 21 bp, Artificial
  Primer sequence for BLSA
gaattccagatccccacagct

SEQ ID NO: 12 - DNA, 21 bp, ARTIFICIAL
  Primer sequence for BLSA
acaccagtatgacccggagtg

SEQ ID NO: 13 - DNA, 20 bp, Artificial
  Primer sequence for BLSA
cgggcctaacagggaattct

SEQ ID NO: 14 - DNA, 20 bp, Artificial
  Primer sequence for BLSA
cccgctgtctgccttttgta

SEQ ID NO: 15 - DNA, 20 bp, Artificial
  Primer sequence for BLSA
cctcccacattgaatccagc

SEQ ID NO: 16 - DNA, 20 bp, Artificial
  Primer sequence for BLSA
gagcagttcctggagcagct

SEQ ID NO: 17 - DNA, 20 bp, Artificial
  Primer sequence for BLSA
tgtgagggacctgtctgcac

SEQ ID NO: 18 - DNA, 19 bp, Artificial
  Primer sequence for BLSA
agtcatcctccgtgtggca

SEQ ID NO: 19 - DNA, 19 bp, Artificial
  Primer sequence for BLSA
ggctgatcctccaaggtcc

SEQ ID NO: 20 - DNA, 19 bp, Artificial
  Primer sequence for BLSA
accagcaggtccccttcaa

SEQ ID NO: 21 - DNA, 22 bp, Artificial
  Primer sequence for BLSA
gtgaacccttccacctgattgt

SEQ ID NO: 22 - DNA, 21 bp, Artificial
  Primer sequence for BLSA
gaccttggaggatcagccagt

SEQ ID NO: 23 - DNA, 20 bp, Artificial
  Primer sequence for BLSA
cgggcctaacagggaattct

SEQ ID NO: 24 - DNA, 20 bp, Artificial
  Primer sequence for BLSA
cccgctgtctgccttttgta

(19) United States
(12) Patent Application Publication
(10) Pub. No.: US 2003/0147887 A1
(43) Pub. Date: Aug. 7, 2003

(54) B-cell lymphoma specific antigen for use in diagnosis and treatment of B-cell malignancies

(76) Inventors: Shen-Wu Wang, Sugar Land, TX (US); Guanghui Hu, Houston, TX (US); Yucheng Li, Houston, TX (US); Zhengbin Yao, Sugar Land, TX (US)

Correspondence Address: TANOX, INC., 10301 STELLA LINK, HOUSTON, TX, 77025, US

(21) Appl. No.: 10/286,927

(22) Filed: Nov. 2, 2002

Related U.S. Application Data
(60) Provisional application No. 60/337,542, filed on Nov. 5, 2001.

(51) Int. Cl. A61K 39/395; C12Q 1/68; G01N 33/574; A61K 39/00; C07K 16/30

(52) U.S. Cl. 424/144.1; 424/185.1; 530/388.22; 435/7.23; 435/6

(57) ABSTRACT

The present invention provides vaccines, antibodies, and diagnostic tools for the diagnosis and/or treatment of B-cell mediated diseases, particularly B-cell lymphomas.

This application claims priority to U.S. Provisional Application No. 60/337,542, filed Nov. 2, 2001.

### FIELD OF THE INVENTION

This invention relates generally to molecules, e.g., peptides and antibodies, that interact with B-cell Lymphoma Specific Antigen (“BLSA”).

### BACKGROUND

Malignant tumors often express characteristic antigens or “markers” which offer a mechanism for tumor prevention, resistance or treatment. The antigens which are characteristic of the tumor may be purified and formulated into vaccines. This may stimulate an antibody response and a cellular immune response which are helpful in controlling tumor growth. At a minimum, the antibodies raised by these antigens can be used as detection tools to monitor the level of lymphoma-associated marker in the host to track the course of the disease, identify patients that have an early stage of the disease that are currently asymptomatic, or to monitor the effectiveness of treatment.

B-cell lymphomas contribute significantly to worldwide cancer mortality. The disease progresses in stages. In the early stage, B-cell lymphoma is often an indolent disease characterized by the accumulation of small mature functionally-incompetent malignant B-cells having a relatively long half-life. Eventually, the malignant B-cell doubling time decreases and patients become increasingly symptomatic. While treatment can provide symptomatic relief, the overall survival of the patients is only minimally affected. In the late stages, the disease is characterized by significant anemia and/or thrombocytopenia. Due to the very low rate of cellular proliferation, B-cell lymphoma of this type is often resistant to current treatments and thus the disease causes death.

Current diagnostic methods for B-cell lymphoma include taking a tissue sample, typically by needle or surgical biopsy, and then analyzing the tissue for cancerous cells. Generally, a blood sample is collected and a pathologist analyzes B-cells for malignancy. The presence of malignant cells indicates that the patient has B-cell lymphoma.

Current methods for treating of B-cell lymphomas depend on the stage and grade of the disease. Adult patients with early-stage disease may be treated with local radiation, with or without chemotherapy. Patients with more advanced but low-grade disease may remain untreated as long as no symptoms or lymphoma-related organ compromise occur, a watch and wait strategy. When treatment becomes necessary, the options typically include single-agent alkylator chemotherapy, low-intensity combined chemotherapy without an anthracycline, and whole-body irradiation. These traditional methods for treating B-cell lymphoma often have limited utility due to toxic side effects. The use of monoclonal antibodies to direct radionuclides, toxins, or other therapeutic agents to the cancer cells present an alternative method for limiting the side effects from the drugs and damage to normal tissue, known as Monoclonal Antibody Therapy.

The monoclonal antibodies by themselves may enhance a patient's immune response to the cancer. Some antitumor effects have been seen in the antibody treatment of lymphoma as well as other cancers. Monoclonal antibodies can also be used in other ways. The antibodies can be bound to a chemotherapy agent and administered in combination. This method permits the chemical from the chemotherapy and the immune response from the antibody to attach the cell. Also, chemotherapy can be more effective when the cells are weakened by the monoclonal antibodies. Further, radiation can be combined with monoclonal antibody therapy. For this method, the monoclonal antibodies contain a radioactive substance such as radioactive iodine that targets and destroys the cancer cells. This method permits the tumor cells receive a large amount of radiation while the normal tissue receives a relatively small amount of radiation. Similarly, radioisotope-labeled monoclonal antibodies may also prove useful in diagnosing certain types of cancer. In addition, monoclonal antibodies may also be linked to other forms of biological response modifiers (BRMs) or toxins. When the linked antibodies bind to cancer cells, they deliver these substances directly to the tumor where it hopefully destroys the cancer cells.

Monoclonal Antibody Therapy has shown promise for the treatment of some types of lymphomas, such as Non-Hodgkin's Lymphoma (NHL). Several monoclonal antibodies are available or in the testing phase, e.g., Rituxan™ (IDEC Pharmaceuticals, Inc., an anti-CD20 antibody), Bexxar™ (Corixa/GlaxoSmithKline, an anti-CD20 antibody with radioactive iodine 131 attached for treatment of NHL), and Oncolym™ (Peregrine Pharmaceuticals, Inc., an anti-HLA-Dr10 antibody with an Iodine 131 radiolabel). Monoclonal antibodies are made by injecting human cancer cells into mice and allowing the murine immune systems to make antibodies against a protein specific to the cancer cells. The cells that make the antibody are collected and fused with an immortal cell to create a hybridoma. These hybridomas produce large quantities of pure monoclonal antibodies that bind the protein specific for that cancer cell. In the case of B-cell lymphomas, the antibodies discussed above are directed against the protein CD20. One disadvantage to this form of therapy is that CD20 is not expressed in pre-B-cell lymphoma, only in mature B-cells.

Thus, there exists a continuing need for new methods for diagnosing and treating the disease, particularly an antigen that is highly expressed in pre-B-cell lymphoma cells. The present invention provides such an antigen, a newly identified B-cell specific protein, BLSA. We have discovered that BLSA is specifically expressed in B-cells and highly upregulated in B-cell lymphoma cell lines, including pre-B-cell lymphoma. BLSA is therefore a new target for the diagnosis and treatment of malignant B-cell diseases, including lymphomas such as NHL and Diffuse Large B-cell Lymphoma (BLBCL).

### SUMMARY OF THE INVENTION

The present invention is directed to the diagnosis and treatment of B-cell mediated disease, including but not limited to B-cell lymphomas, such as low grade/follicular non-Hodgkin's lymphoma (NHL), small lymphocytic (SL) NHL, intermediate grade/follicular NHL, intermediate grade diffuse NHL, high grade inimunoblastic NHL, high grade lymphoblastic NHL, high grade small non-cleaved cell NHL, bulky disease NHL, Diffuse Large B-cell Lymphoma (BLBCL), lymphoplasmacytic lymphoma and Waldenstrom's Macroglobulinemia. Treatment of these abnormal B-cell diseases may be performed alone or in combination with currently used treatment, e.g., cytokines, radiotherapy, mycloablative therapy, and chemotherapy.

One aspect of the invention includes the production and administration of vaccines directed to BLSA for the treatment of B-cell lymphoma or other B-cell mediated diseases.

Another aspect of the invention includes nucleotide constructs that encode BLSA expression of protein in vivo to generate an immune response in the patient, or for generating a protein antigen for making polyclonal or monoclonal antibodies. The invention also includes nucleotide constructs for modulating the expression of BLSA. These nucleotide sequences may be in the form of an expression vector, an antisense construct, a conjugate, or an epitope containing fragment.

Another aspect of the invention includes providing compounds that interact with B cell lymphoma specific antigen (“BLSA”). The interaction can be used to diagnose the presence of BLSA and its presence can be correlated to the presence of or likelihood of the patient developing a B-cell mediated disease. The interaction can be used to treat patients diagnosed as suffering from a B-cell mediated disease by using the interaction to kill the cell or make the cell more susceptible to death when treated by other therapies. Antibodies that interact with BLSA can be used to diagnose or treat a B-cell mediated disease. Other compounds include small molecules that bind to BLSA modulating its expression and/or function.

Another aspect of the invention includes screening for agonists or antagonists that interact with BLSA.

Another aspect of the invention includes methods for immunizing a patient against B-cell lymphoma or other B-cell mediated diseases and antigen constructs useful in such methods.

Other and further objects, features, and advantages of the present invention will be readily apparent to those skilled in the art.

### DETAILED DESCRIPTION OF THE INVENTION

### Definitions

The term “B-cell Lymphoma Specific Antigen” or “BLSA” means a polypeptide having the sequence shown in SEQ ID NO: 1 or its naturally occurring variants.

The term “B-cell lymphoma” means one or more of the malignant diseases of B-cells that is characterized by the presence of BLSA and in particular the presence of elevated levels of BLSA.

The term “variant” means an amino acid sequence that differs from BLSA by one or more amino acids, including modifications, substitutions, insertions, and deletions, and either has the same or similar biological function as BLSA.

The term “agonist” means any molecule that promotes, enhances, or stimulates the normal function of BLSA or its expression. One type of agonist is a molecule that interacts with BLSA in a way that mimics its ligand, including an antibody or antibody fragment.

The term “antagonist” means any molecule that blocks, prevents, inhibits, or neutralizes the normal function of BLSA or its expression. One type of antagonist is a molecule that interferes with the interaction between BLSA and its ligand, including an antibody or antibody fragment. Another type of antagonist is an antisense nucleotide that inhibits proper transcription of native BLSA activating receptor.

The term “antisense” as used herein, refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

The term “knockout” refers to partial or complete reduction of the expression of at least a portion of a polypeptide encoded by an endogenous gene (such as BLSA) of a single cell, selected cells, or all of the cells of a mammal. The mammal may be a “heterozygous knockout” having one allele of the endogenous gene disrupted or “homozygous knockout” having both alleles of the endogenous gene disrupted.

The term “antibody fragment” is a portion of an antibody, such as F(ab′)₂, F(ab)₂, Fab′, Fab, and the like. Regardless of structure, an antibody fragment binds with the same antigen that is recognized by the intact antibody. For example, an anti-BLSA monoclonal antibody fragment binds with an epitope of BLSA. The term “antibody fragment” also includes any synthetic or genetically engineered protein that acts like an antibody by binding to a specific antigen to form a complex. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (“sFv proteins”), and minimal recognition units consisting of the amino acid residues that mimic the hypervariable region.

This invention is not limited to the particular methodology, protocols, cell lines, vectors, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

### The Invention

The invention provides B cell lymphoma vaccines, antibodies specific for BLSA, and diagnostic tools. The nucleic acid sequence of BLSA is depicted in SEQ ID NO 1 and the amino acid sequence is depicted in SEQ ID NO 2.

The active ingredient of the B cell lymphoma vaccine is the B cell lymphoma-specific antigen BLSA, or a fragment thereof, having at least one epitope. The B cell lymphoma associated antigen may be obtained by purification from cells, tissues, the lymphoma itself, or may be synthesized using recombinant techniques. Because BLSA is a native proteins in humans, vaccine constructs of BLSA may contain, e.g., T-cell epitopes or other antigenic aids to break the hosts immune tolerance to the antigen.

Antibodies specific for BLSA may be made by conventional methods, such as cell-to-cell fusion for the production of monoclonal antibodies as disclosed by Kohler and Milstein (Nature (London), 256: 495, 1975), but may be polyclonal or monoclonal, including chimeric, humanized, human, deimmunized, bispecific and heteroconjugate antibodies. Antibodies may also be made recombinantly. Antibodies may be administered for treatment or used in diagnostic methods. The antibodies can be used for therapeutic purposes, by themselves, in complement mediated lysis, or coupled to toxins or therapeutic moieties, such as ricin, cytokines, etc.

Diagnostic tools include assays and kits to monitor the level of lymphoma-associated marker in the host to track the course of the disease, identify patients that have an early asymptomatic stage of the disease, or to monitor the effectiveness of treatment.

Having now generally described the invention, the same will be further understood by reference to more detailed description and certain specific examples which are included herein for purposes of illustration only and are not intended to be limiting unless otherwise specified.

### Diagnostic Tools

We have discovered a new target for screening or diagnosing patients that suffer from B-cell mediated lymphomas. BLSA is very highly expressed in B-cell lymphomas and particularly in pre-B cell lymphoma.(See Tables 1 and 2, infra).

Immunophenotypic characterization of lymphomas by monoclonal antibodies has proved to be a valuable adjunct to histologic diagnosis and has facilitated understanding of the lineage of certain lymphomas. Monoclonal antibodies detecting various antigens have been used or proposed for a number of purposes in research and for diagnostic studies of leukemias and lymphomas in men and animals. The techniques employed include, but are not limited to:

1. Leukocyte identification by phenotype, utilizing flow cytometry, immunofluorescence, immunoenzyme techniques, or immuno electron microscopy.

2. Leukocyte separation techniques, including flow cytometry and panning.

3. Identification and classification of lymphomas.

4. Radioimmunimaging of lymphomas in animals and man.

5. Radioimmunotherapy of lymphomas in animals and man.

6. Studies of leukocyte differentiation, maturation and function in experimental models and human disease.

Diagnostic antigen-antibody reactions can be detected by a variety a methods known in the art, using markers to label either the antibody or the antigen. Commonly used markers are chromogens, such as fluorochromes, enzymes, radioactive and radiopaque compounds. Fluorochromes are dyes that absorb radiation, for example ultraviolet light, are excited by it and as a result, emit visible light. Fluorochromes that are useful as markers are capable of forming covalent bonds with protein molecules and having a high fluorescence emission in the visible spectrum with a color different from that of tissues. Commonly used fluorochromes are fluorescein isothiocyanate (FITC) and tetramethylrhodamine isothiocyanate (TRITC).

The methods that use antibodies labeled with fluorochrome markers are usually referred to as immunofluorescence methods. In the so called “direct method” fluorochrome-labeled antibody is applied to the preparation containing the corresponding antigen. In the “indirect method” the antigen is treated with its corresponding unlabeled antibody, and the resultant antigen-antibody complex is treated with a fluorochrome-labeled antibody to the immunoglobulin of the animal species that provided the unlabeled antibody used in the first step. In diagnostic immunology, the antigen-containing substrate may be incubated with the patient's serum, and then with fluorochrome-labeled mouse, rabbit or goat antibody to human immunoglobulins. The indirect method can provide higher sensitivity. For detecting immunofluorescent specimens, fluorescence microscopes, that are simple modifications of standard transmitted light microscopes, can be used. If necessary, the results may be recorded by photomicrography.

Enzymes may also be used as labels if, on interaction with their substrate, they form a detectable precipitant or visible emission. Immunoenzyme procedures can be used to localize antigens with the aid of enzyme-labeled antibodies. Several enzymes have been employed as markers, such as horseradish peroxidase and alkaline phosphatase. A widely used protocol for the detection of antigens by enzyme-linked antibodies is referred to as Enzyme Linked Immunosorbent Assay (ELISA) that may be performed as a direct method or in sandwich format.

As radioactive markers, any of the well-known medical radionuclides can be used. Suitable radionuclides include Tc-99m, 1-123, In-111, In-113m, Ga-67, or other suitable gamma-emitters. The radionuclides can be conjugated to the monoclonal antibody of the present invention by conventional techniques. Iodination, for example, may be accomplished using the chloramine-T method described by S. Mills, et al. .sup.123 I-Radiolabeling of Monoclonal Antibodies for In Vivo Procedures, Hybridoma 5, 265-275 (1986). This technique may be used to effect iodination to render the antibody radiopaque, or to attach a radionuclide, such as I-125 or I-131. Other radionuclides may be attached to the antibody through chelation with benzyl EDTA or DPTA conjugation procedures. Still other suitable techniques include the iodogen method disclosed by M. Pimm, et al., In Vivo Localization of Anti-Osteogenic Sarcoma 791T Monoclonal Antibody, Int. J. Cancer. 30, 75 (1982), and direct iodination with radioactive sodium iodide.

Radiopaque materials suitable for labeling antibodies include iodine compounds, barium compounds, gallium compounds, thallium compounds, and the like. Specific examples of radiopaque materials include barium, diatnzoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, acid, iosulamide meglumine, iosumetic acid, iotasul, iotetric acid, iothalamic acid, iotroxic acid, ioxaglic acid, ioxotrizoic acid, ipodate, meglumine, metrizamide, metrizoate, propylidone, and thallous chloride.

In another aspect, the invention relates to methods for imaging lesions. A method for imaging lesions characteristic of certain lymphomas, may comprise the steps of: obtaining monoclonal antibody specific to BLSA; labeling said antibody; contacting said labeled antibody with a biological sample obtained from a mammal; and imaging said label. For this purpose, the anti-BLSA antibody may be labeled. Suitable labels include, for example, radiolabels, radiopaque materials, and magnetic resonance-enhancing materials. The radiolabels and radiopaque materials have been discussed above. Suitable techniques for imaging labels localized in tissues expressing antibody are known in the art. For example, if the label is a gamma-emitting radionuclide, suitable imaging techniques include gamma cameras and single photon emission computed tomography (SPECT) techniques. If the antibody has been labeled with a radiopaque material, radiographic imaging may be applied. Other suitable techniques include computed axial tomography (CAT) scans, fluoroscopy and conventional X-ray imaging.

Materials that can be detected by or that enhance the effects of magnetic resonance imaging equipment also may be conjugated to the antibodies. Suitable conventional magnetic resonance-enhancing compounds include gadolinium, copper, iron, and chromium. These metal atoms may be prepared in the form of conventional organometallic chelates, which are then bound to the antibody. The foregoing methods along with other routine techniques of immunodiagnosis are disclosed in standard laboratory textbooks. See, for example, Rose, N. R. and Pierluigi, E. B. in Methods in Immunodiagnosis, Second Edition, John Wiley & Sons, Publishers, New York, Chichester, Brisbane, Toronto, 1980; Current Protocols in Molecular Biology, Green Publishing Associates and Wiley-Interscience, 1987.

The present invention also provides methods for detecting the presence of or elevated levels of BLSA in a patient. The method is useful for determining whether a patient is suffering from B-cell lymphoma, for monitoring the progression or stage of the disease, or monitoring the effectiveness of treatment of the disease. The method comprises collecting a sample from a patient; exposing the sample to a molecule that interacts with BLSA; and detecting the presence of an interaction between BLSA and the molecule or measuring the the amount of product formed.

The sample can be any biological fluid or tissue that contains B-cells, including blood. The sample is collected by any well known means, such as biopsy or simply drawing blood from the patient.

The molecule that interacts with BLSA can be, e.g., a small molecule, a protein, a peptide, an antibody, an oligonucleotide or a ligand. Preferably the molecule is an antibody that specifically bind to BLSA. The interaction between the molecule and BLSA can be detected by any well know means, e.g., fluorometry, chemiluminescence, ELISA, FACS analysis, solid-phase RIA, etc. When the molecule is an antibody that binds to BLSA, the preferred method of detection is ELISA.

The method of detecting the level of expression of BLSA may include measurement by PCR, e.g., real time quantitative PCR. This method may be performed using oligonucleotide primers such as:

| | |
|---|---|
| F: CAGAGCCCCCAGCTAGAGATC | (SEQ ID NO 3) |
| R: GTGCAGCAGAGCTGGAAGC | (SEQ ID NO 4) |
| F: GCAGTGGCATCTTCCAGAGC | (SEQ ID NO 5) |
| R: CAGATGCTGTTTCTGGGATCC | (SEQ ID NO 6) |
| F: GATCAGAGTGCAGGGTGCTTC | (SEQ ID NO 7) |
| R: GGATTCAATGTGGGAGGTGC | (SEQ ID NO 8) |
| F: GTGAGGGACCTGTCTGCACTG | (SEQ ID NO 9) |
| R: AGTCATCCTCCGTGTGGCA | (SEQ ID NO 10) |
| F: GAATTCCAGATCCCCACAGCT | (SEQ ID NO 11) |
| R: ACACCAGTATGACCCGGAGTG | (SEQ ID NO 12) |
| F: CGGGCCTAACAGGGAATTCT | (SEQ ID NO 13) |
| R: CCCGCTGTCTGCCTTTTGTA | (SEQ ID NO 14) |
| F: CCTCCCACATTGAATCCAGC | (SEQ ID NO 15) |
| R: GAGCAGTTCCTGGAGCAGCT | (SEQ ID NO 16) |
| F: TGTGAGGGACCTGTCTGCAC | (SEQ ID NO 17) |
| R: AGTCATCCTCCGTGTGGCA | (SEQ ID NO 18) |
| F: GGCTGATCCTCCAAGGTCC | (SEQ ID NO 19) |
| R: ACCAGCAGGTCCCCTTCAA | (SEQ ID NO 20) |

Other primer sets can be readily determined by one skilled in the art by well known techniques. The method may also include the measurement of the relative expression of BLSA by comparing the expression level in the patient to that of a normal tissue.

Also included in the invention are diagnostic kits comprising, e.g., an antibody specific for BLSA or primers for detecting expression levels of BLSA.

### Agonists and Antagonists

In another aspect, the present invention provides agonists and antagonists that specifically bind to BLSA and inhibit or activate its expression or action. Types of agonist and antagonists include, but are not limited to, polypeptides, proteins, peptides, glycoproteins, glycopeptides, glycolipids, polysaccharides, oligosaccharides, nucleotides, organic molecules, bioorganic molecules, peptidomimetics, pharmacological agents and their metabolites, and transcriptional and translation control sequences.

In one embodiment, the agonists and antagonists are antisense oligonucleotides or other nucleic acid constructs that can be used to modulate the function of nucleic acid molecules encoding BLSA, ultimately modulating the amount of BLSA produced. This is accomplished by providing antisense compounds, which specifically hybridize with one or more nucleic acids encoding BLSA. The specific hybridization of an oligomeric compound with its target nucleic acid interferes with the normal function of the nucleic acid. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of BLSA. In the context of the present invention, “modulation” means either an increase (stimulation) or a decrease (inhibition) in the expression of a gene. In the context of the present invention, inhibition is the preferred form of modulation of gene expression and mRNA is a preferred target. “Targeting” an antisense compound to BLSA includes determination of a site or sites within this gene for the antisense interaction to occur such that the desired effect, e.g., detection or modulation of expression of the protein, will result. A preferred intragenic site is the region encompassing the translation initiation or termination codon of BLSA open reading frame (ORF). The methodology for antisense technology is disclosed, for example, in Crooke ST: Basic Principles of antisense technology. In Antisense Drug Technology—Principles, Strategies and Applications. Edited by Crooke ST. New York: Marcel Dekker, Inc.; 2001: 1-28.

In another embodiment, the antagonists can be small interfering RNAs (siRNA) by RNA interference (RNAi) process, which uses short (generally 2123 bp) double-stranded RNA to target BLSA for degradation, thereby silencing its expression. The siRNA duplexes can be designed according to the guidelines as described (Elbashir, S M, Harborth J, Lendeckel W, Yalcin A, Weber K, and Tuschl T. (2001). Duplexes of 21-nucleotide RNAs mediate RNA interference in cultured mammalian cells. Nature 411, 494-498), and used in multiple ways such as a viral-mediated strategy (Xia, H. et al. (2002) siRNA-mediated gene silencing in vitro and in vivo. Nature Biotechnology 20: 1006).

Agonists and antagonists may be antibodies that bind specifically to BLSA and influence its biological actions and/or functions, e.g., to activate or inhibit the production of BLSA. The antibodies can be polyclonal or monoclonal antibodies but are preferably monoclonal antibodies.

Agonist antibodies are used to prevent or treat diseases characterized by relatively low levels of BLSA expression compared to non-disease states. Antagonist antibodies are used to prevent or treat diseases characterized by relatively high levels of BLSA expression compared to non-disease states.

The agonists and antagonists and methods of the present invention may be used to treat a variety of B-cell lymphomas, including low grade/follicular non-Hodgkin's lymphoma (NHL), small lymphocytic (SL) NHL, intermediate grade/follicular NHL, intermediate grade diffuse NHL, high grade inimunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention.

For instance, a recent classification system proposed by European and American pathologists is called the Revised European American Lymphoma (REAL) Classification. This classification system recognizes Mantle cell lymphoma and Marginal cell lymphoma among other peripheral B-cell neoplasms, and separates some classifications into grades based on cytology, i.e., small cell, mixed small and large, large cell. It will be understood that all such classified lymphomas may benefit from the combined therapies of the present invention.

The U.S. National Cancer Institute (NCI) has in turn divided some of the REAL classes into more clinically useful “indolent” or “aggressive” lymphoma designations. Indolent lymphomas include follicular cell lymphomas, separated into cytology “grades,” diffuse small lymphocytic lymphoma/chronic lymphocytic leukemia (CLL), lymphoplasmacytoid/Waldenstrom's Macroglobulinemia, Marginal zone lymphoma and Hairy cell leukemia. Aggressive lymphomas include diffuse mixed and large cell lymphoma, Burkitt's lymphoma/diffuse small non-cleaved cell lymphoma, Lymphoblastic lymphoma, Mantle cell lymphoma and AIDS-related lymphoma. These lymphomas may also benefit from the combined therapeutic regimens of the present invention.

Non-Hodgkin's lymphoma has also been classified on the basis of “grade” based on other disease characteristics including low-grade, intermediate-grade and high-grade lymphomas. Low-grade lymphoma usually presents as a nodal disease, and is often indolent or slow-growing. Intermediate- and high-grade disease usually presents as a much more aggressive disease with large extranodal bulky tumors. Intermediate- and high-grade disease, as well as low grade NHL, may benefit from the combined therapeutic regimens of the present invention.

Antibodies specific to BLSA may also be conjugated to other compounds that kill the diseased cells directly, make the diseased cells more susceptible to killing, e.g., phagocytosis, or cause diseased cells to undergo apoptosis. The antibody may be operatively attached to, e.g., a chemotherapeutic agent, a radiotherapeutic agent, an anti-angiogenic agent such as angiopoictin, angiostatin, vasculostatin, canstatin or maspin, an apoptosis-inducing agent, a steroid, an antimetabolite, an anthracycline, a vinca alkaloid, an anti-tubulin drug, such as colchicine, taxol, vinblastine, vincristine, vindescine and a combretastatin, an antibiotic, a cytokine, an alkylating agent or coagulant., a cytotoxic, cytostatic or anticellular agent capable of killing or suppressing the growth or cell division of lymphoma cells, a plant-, fungus- or bacteria-derived toxin, such as ricin A chain, deglycosylated ricin A chain, a ribosome inactivating protein, alpha.-sarcin, gelonin, aspergillin, restrictocin, a ribonuclease, an epipodophyllotoxin, diphtheria toxin or Pseudomonas exotoxin.

The dosages of BLSA agonist or antagonist vary according to the age, size, and character of the particular mammal and the disease. Skilled artisans can determine the dosages based upon these factors. The agonist or antagonist can be administered in treatment regimes consistent with the disease, e.g., a single or a few doses over a few days to ameliorate a disease state or periodic doses over an extended time to prevent allergy or asthma.

The agonists and antagonists can be administered to the mammal in any acceptable manner including by injection, using an implant, and the like. Injections and implants are preferred because they permit precise control of the timing and dosage levels used for administration. The agonists and antagonists are preferably administered subcutaneously, but may be by intravenous, intramuscular, or intraperitoneal injection, or by subcutaneous implant.

When administered by injection, the agonists and antagonists can be administered to the mammal in a injectable formulation containing any biocompatible and agonists and antagonists compatible carrier such as various vehicles, adjuvants, additives, and diluents. Aqueous vehicles such as water having no nonvolatile pyrogens, sterile water, and bacteriostatic water are also suitable to form injectable solutions. In addition to these forms of water, several other aqueous vehicles can be used. These include isotonic injection compositions that can be sterilized such as sodium chloride, Ringer's, dextrose, dextrose and sodium chloride, and lactated Ringer's. Nonaqueous vehicles such as cottonseed oil, sesame oil, or peanut oil and esters such as isopropyl myristate may also be used as solvent systems for the compositions. Additionally, various additives which enhance the stability, sterility, and isotonicity of the composition including antimicrobial preservatives, antioxidants, chelating agents, and buffers can be added. Any vehicle, diluent, or additive used would, however, have to be biocompatible and compatible with the agonists and antagonists according to the present invention.

### Antibody and Antibody Production

In another aspect, the present invention provides an antibody that binds to the BLSA of the present invention and methods for producing such antibody, including antibodies that function as native BLSA agonists or antagonists. In one embodiment, the method comprises using isolated BLSA or antigenic fragments thereof as an antigen for producing antibodies that bind to the BLSA of the present invention in a known protocol for producing antibodies to antigens, including polyclonal and monoclonal antibodies. In another embodiment, the method comprises using host cells that express recombinant BLSA as an antigen. In a further embodiment, the method comprises using DNA expression vectors containing the BLSA gene to express BLSA as an antigen for producing the antibodies.

Methods for producing antibodies, including polyclonal, monoclonal, monovalent, humanized, human, bispecific, and heteroconjugate antibodies, are well known to skilled artisans.

### Polyclonal Antibodies

Polyclonal antibodies can be produced in a mammal by injecting an immunogen alone or in combination with an adjuvant. Typically, the immunogen is injected in the mammal using one or more subcutaneous or intraperitoneal injections. The immunogen may include the polypeptide of interest or a fusion protein comprising the polypeptide and another polypeptide known to be immunogenic in the mammal being immunized. The immunogen may also include cells expressing a recombinant vector or a DNA expression vector containing the BLSA gene. Examples of such immunogenic proteins include, but are not limited to, keyhole limpet hemocyanin, serum albumin, bovine thyroglobulin, and soybean trypsin inhibitor. Examples of adjuvants include, but are not limited to, Freund's complete adjuvant, MPL-TDM adjuvant (monophosphoryl Lipid A, synthetic trehalose dicorynomycolate), and CpG related oligonucleotides. The immunization protocol may be selected by one skilled in the art without undue experimentation.

### Monoclonal Antibodies

Monoclonal antibodies can be produced using hybridoma methods such as those described by Kohler and Milstein, Nature, 256:495 (1975). In a hybridoma method, a mouse, hamster, or other appropriate host mammal, is immunized with an immunogen to elicit lymphocytes that produce or are capable of producing antibodies that will specifically bind to the immunogen. Alternatively, the lymphocytes may be immunized in vitro. The immunogen will typically include the polypeptide of interest or a fusion protein containing such polypeptide. Generally, peripheral blood lymphocytes (“PBLs”) cells are used if cells of human origin are desired. Spleen cells or lymph node cells are used if cells of non-human mammalian origin are desired. The lymphocytes are then fused with an immortalized cell line using a suitable fusing agent, e.g., polyethylene glycol, to form a hybridoma cell (Goding, Monoclonal Antibodies: Principles and Practice, pp 59-103 (Academic Press, 1986)). Immortalized cell lines are usually transformed mammalian cells, particularly rodent, bovine, or human myeloma cells. Usually, rat or mouse myeloma cell lines are employed. The hybridoma cells may be cultured in a suitable culture medium that preferably contains one or more substances that inhibit the growth or survival of the unfused immortalized cells. For example, if the parental cells lack the enzyme hypoxanthine guanine phosphoribosyl transferase (HGPRT), the culture medium for the hybridomas typically will include hypoxanthine, aminopterin, and thymidine (HAT medium). The HAT medium prevents the growth of HGPRT deficient cells.

Preferred immortalized cell lines are those that fuse efficiently, support stable high level expression of antibody by the selected antibody producing cells, and are sensitive to a medium such as HAT medium. More preferred immortalized cell lines are murine myeloma lines such as those derived from MOPC-21 and MPC-11 mouse tumors available from the Salk Institute Cell Distribution Center, San Diego, Calif. USA, and SP2/0 or X63-Ag8-653 cells available from the American Type Culture Collection, Rockville, Md. USA. Human mycloma and mouse-human heteromyeloma cell lines also have been described for use in the production of human monoclonal antibodies (Kozbor, J. Immunol. 133:3001 (1984); Brodeur et al., Monoclonal Antibody Production Techniques and Applications, pp. 51-63 (Marcel Dekker, Inc., New York, 1987)). The mouse myeloma cell line NSO may also be used (European Collection of Cell Cultures, Salisbury, Wiltshire UK). Human myeloma and mouse-human heteromyeloma cell lines, well known in the art, can also be used to produce human monoclonal antibodies.

The culture medium used for culturing hybridoma cells can then be assayed for the presence of monoclonal antibodies directed against the polypeptide of interest. Preferably, the binding specificity of monoclonal antibodies produced by the hybridoma cells is determined by immunoprecipitation or by an in vitro binding assay, e.g., radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods. Suitable culture media for this purpose include Dulbecco's Modified Eagle's Medium and RPMI-1640 medium. Alternatively, the hybridoma cells may be grown in vivo as ascites in a mammal.

The monoclonal antibodies secreted by the subclones are isolated or purified from the culture medium or ascites fluid by conventional immunoglobulin purification procedures such as protein A-Sepharose, hydroxylapatite chromatography, gel electrophoresis, dialysis, or affinity chromatography.

The monoclonal antibodies may also be produced by recombinant DNA methods, e.g., those described in U.S. Pat. No. 4,816,567. DNA encoding the monoclonal antibodies of the invention can be readily isolated and sequenced using conventional procedures, e.g., by using oligonucleotide probes that are capable of binding specifically to genes encoding the heavy and light chains of murine antibodies (Innis M. et al. In “PCR Protocols. A Guide to Methods and Applications”, Academic, San Diego, Calif. (1990), Sanger, F. S, et al. Proc. Nat. Acad. Sci. 74:5463-5467 (1977)). The hybridoma cells described herein serve as a preferred source of such DNA. Once isolated, the DNA may be placed into expression vectors. The vectors are then transfected into host cells such as simian COS cells, Chinese hamster ovary (CHO) cells, or myeloma cells that do not otherwise produce immunoglobulin protein. The recombinant host cells are used to produce the desired monoclonal antibodies. The DNA also may be modified, for example, by substituting the coding sequence for human heavy and light chain constant domains in place of the homologous murine sequences or by covalently joining the immunoglobulin coding sequence to all or part of the coding sequence for a non-immunoglobulin polypeptide. Such a non-immunoglobulin polypeptide can be substituted for the constant domains of an antibody or can be substituted for the variable domains of one antigen combining site of an antibody to create a chimeric bivalent antibody.

Monovalent antibodies can be produced using the recombinant expression of an immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. Similarly, in vitro methods can be used for producing monovalent antibodies. Antibody digestion can be used to produce antibody fragments, preferably Fab fragments, using known methods.

Antibodies and antibody fragments can be produced using antibody phage libraries generated using the techniques described in McCafferty, et al., Nature 348:552-554 (1990). Clackson, et al., Nature 352:624-628 (1991) and Marks, et al., J. Mol. Biol. 222:581-597 (1991) describe the isolation of murine and human antibodies, respectively, using phage libraries. Subsequent publications describe the production of high affinity (nM range) human antibodies by chain shuffling (Marks, et al, Bio/Technology 10:779-783 (1992)), as well as combinatorial infection and in vivo recombination as a strategy for constructing very large phage libraries (Waterhouse, et al., Nuc. Acids. Res. 21:2265-2266 (1993)). Thus, these techniques are viable alternatives to traditional monoclonal antibody hybridoma techniques for isolation of monoclonal antibodies. Also, the DNA may be modified, for example, by substituting the coding sequence for human heavy-chain and light-chain constant domains in place of the homologous murine sequences (U.S. Pat. No. 4,816,567; Morrison, et al., Proc. Nat. Acad. Sci. USA 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Antibodies can also be produced using use electrical fusion rather than chemical fusion to form hybridomas. This technique is well established. Instead of fusion, one can also transform a B-cell to make it immortal using, for example, an Epstein Barr Virus, or a transforming gene “Continuously Proliferating Human Cell Lines Synthesizing Antibody of Predetermined Specificity,” Zurawaki, V. R. et al, in “Monoclonal Antibodies,” ed. by Kennett R. H. et al, Plenum Press, N.Y. 1980, pp 19-33.

### Humanized Antibodies

Humanized antibodies can be produced using the method described by Winter in Jones et al., Nature, 321:522-525 (1986); Riechmann et al., Nature, 332:323-327 (1988); and Verhoeyen et al., Science, 239:1534-1536 (1988). Humanization is accomplished by substituting rodent CDRs or CDR sequences for the corresponding sequences of a human antibody. Generally, a humanized antibody has one or more amino acids introduced into it from a source that is non-human. Such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Humanized forms of non-human (e.g., murine or bovine) antibodies are chimeric immunoglobulins, immunoglobulin chains, or immunoglobulin fragments such as Fv, Fab, Fab′, F(ab′)₂, or other antigen-binding subsequences of antibodies that contain minimal sequence derived from non-human immunoglobulin. Humanized antibodies include human immunoglobulins (recipient antibody) wherein residues from a complementary determining region (CDR) of the recipient are replaced by residues from a CDR of a non-human species (donor antibody) such as mouse, rat, or rabbit having the desired specificity, affinity, and capacity. Sometimes, Fv framework residues of the human immunoglobulin are replaced by corresponding non-human residues. Humanized antibodies also comprise residues that are found neither in the recipient antibody nor in the imported CDR or framework sequences. In general, humanized antibodies comprise substantially all oR at least one and typically two variable domains wherein all or substantially all of the CDR regions correspond to those of a non-human immunoglobulin and all or substantially all of the FR regions are those of a human immunoglobulin consensus sequence. Humanized antibodies optimally comprise at least a portion of an immunoglobulin constant region (Fc), typically that of a human immunoglobulin.

### Human Antibodies

Human antibodies can be produced using various techniques known in the art, e.g., phage display libraries as described in Hoogenboom and Winter, J. Mol. Biol., 227:381 (1991) and Marks et al., J. Mol. Biol., 222:581 (1991). Human monoclonal antibodies can be produced using the techniques described in Cole et al., Monoclonal Antibodies and Cancer Therapy, Alan R. Liss, p. 77 (1985) and Boemer et al., J. Immunol., 147(1):86-95 (1991). Alternatively, transgenic animals, e.g., mice, are available which, upon immunization, can produce a full repertoire of human antibodies in the absence of endogenous immunoglobulin production. Such transgenic mice are available from Abgenix, Inc., Fremont, Calif., and Medarex, Inc., Annandale, N.J. It has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immunol. 7:33 (1993); and Duchosal et al. Nature 355:258 (1992). Human antibodies can also be derived from phage-display libraries (Hoogenboom et al., J. Mol. Biol. 227:381 (1991); Marks et al., J. Mol. Biol. 222:581-597 (1991); Vaughan, et al., Nature Biotech 14:309 (1996)).

### Bispecific Antibodies

Bispecific antibodies can be produced by the recombinant co-expression of two immunoglobulin heavy-chain/light-chain pairs wherein the two heavy chains have different specificities. Bispecific antibodies are monoclonal, preferably human or humanized, antibodies that have binding specificities for at least two different antigens. In the present invention, one of the binding specificities is for the BLSA and the other is for any other antigen, preferably a cell surface receptor or receptor subunit. Because of the random assortment of immunoglobulin heavy and light chains, these hybridomas produce a potential mixture of ten different antibodies. However, only one of these antibodies has the correct bispecific structure. The recovery and purification of the correct molecule is usually accomplished by affinity chromatography.

Antibody variable domains with the desired binding specificities (antibody-antigen combining sites) can be fused to immunoglobulin constant domain sequences. The fusion preferably is with an immunoglobulin heavy chain constant domain comprising at least part of the hinge, CH2, and CH3 regions. Preferably, the first heavy-chain constant region (CH1) containing the site necessary for light-chain binding is present in at least one of the fusions. DNAs encoding the immunoglobulin heavy-chain and, if desired, the immunoglobulin light chain is inserted into separate expression vectors and co-transfected into a suitable host organism. Suitable techniques are shown in for producing bispecific antibodies are described in Suresh et al., Methods in Enzymology, 121:210 (1986).

### Heteroconjugate Antibodies

Heteroconjugate antibodies can be produced known protein fusion methods, e.g., by coupling the amine group of one an antibody to a thiol group on another antibody or other polypeptide. If required, a thiol group can be introduced using known methods. For example, immunotoxins comprising an antibody or antibody fragment and a polypeptide toxin can be produced using a disulfide exchange reaction or by forming a thioether bond. Examples of suitable reagents for this purpose include iminothiolate and methyl-4-mercaptobutyrimidate. Such antibodies can be used to target immune system cells to unwanted cells or to treat HIV infections.

### Polynucleotides

In another aspect, the present invention provides an isolated polynucleotide comprising a nucleotide sequence selected from the group consisting of SEQ ID NO:1; a variant of SEQ ID NO:1; a fragment of SEQ ID NO:1; a nucleotide sequence that encodes a polypeptide having the amino acid sequence selected from the group consisting of SEQ ID NO:2; a variant of SEQ ID NO:2; and a fragment of SEQ ID NO:2.

The isolated polynucleotides of the present invention are preferably coding sequences for BLSA. In one aspect of the invention, polynucleotides are used to produce BLSA that function as antigens in the process used to produce the agonist and antagonist antibodies that specifically bind to BLSA and inhibit or activate the expression or action of BLSA function. In another aspect of the invention, polynucelotides may be used as vaccines for DNA immunization techniques. Various other methods described utilizing polynucleotides for the expression of BLSA are also contemplated.

### Vectors and Host Cells

In another aspect, the present invention provides a vector comprising a nucleotide sequence encoding the BLSA of the present invention and a host cell comprising such a vector.

By way of example, the host cells may be mammalian cells, (e.g. CHO cells), prokaryotic cells (e.g., E. coli) or yeast cells (e.g., Saccharomyces cerevisiae). A process for producing vertebrate fused polypeptides is further provided and comprises culturing host cells under conditions suitable for expression of vertebrate fused and recovering the same from the cell culture.

### Vaccines

An ideal way of treating a disease caused by a malfunction of the immune system in distinguishing self from foreign, would be by encouraging this system to elicit self protective immunity and thus restrain its own harmful reactivity to times when such a response is needed. This task has been achieved by using DNA vaccination. DNA inoculation represents an approach to vaccine and immune therapeutic development. DNA vaccines represent a novel means of expressing antigens in vivo for the generation of both humoral and cellular immune responses. This technology has proven successful in obtaining immunity not only to foreign antigens and tumors, but also to self antigens, such as a T cell receptor genes or autologous cytokines. Since DNA vaccination elicits both cellular and humoral responses against products of a given construct, it can be a very effective tool in eradicating diseased cells. The direct injection of gene expression cassettes into a living host transforms a number of cells into factories for production of the introduced gene products. Expression of these delivered genes has important immunological consequences and may result in the specific immune activation of the host against the novel expressed antigens. This unique approach to immunization can overcome deficits of traditional antigen-based approaches and provide safe and effective prophylactic and therapeutic vaccines. The host normal cells (nonhemopoietic) can express and present the tumor antigens to the immune system. The transfected cells display fragments of the antigens on their cell surfaces together with class I or class II major hisotcompatibility complexes (MHC I, MHC II). The MHC I display acts as a distress call for cell-mediated immune response, which dispatches CTLs that destroy the transfected cells. The CTLs are important for tumor regressions. In general, when a cytopathic virus infects a host normal cell, the viral proteins are endogenously processed and presented on the cell surface, or in fragments by MHC molecules. Foreign defined nucleic acid transfected and expressed by normal cells can mimic viral infections.

An immunogenic fusion polypeptide encoded on a vector as described herein comprises a T cell epitope portion and a B cell epitope portion. A T cell epitope portion encoded on the vector of this comprises a broad range or “universal” helper T cell epitope which bind the antigen presenting site of multiple (i.e., 2, 3, 4, 5, 6 or more) class II major histocompatibility (MHC) molecules and can form a tertiary complex with a T cell antigen receptor, i.e., MHC:antigen:T cell antigen receptor. By “non-endogenous protein” is meant a protein which is not the endogenous to the individual who is to be treated. Such non-endogenous proteins, or fragments thereof, useful as T cell epitope portions of the immunogenic fusion polypeptide include tetanus toxoid; diphtheria toxin; class II MHC-associated invariant chain; influenza hemagglutinin T cell epitope; keyhole limpet hemocyanin (KLH); a protein from known vaccines including pertussis vaccine, the Bacile Calmette-Guerin (BCG) tuberculosis vaccine, polio vaccine, measles vaccine, mumps vaccine, rubella vaccine, and purified protein derivative (PPD) of tuberculin; and also synthetic peptides which bind the antigen presenting site of multiple class II histocompatibility molecules, such as those containing natural amino acids described by Alexander et al. (Immunity, 1: 751-761 (1994)). When attached to a BLSA B cell epitope portion, the T cell epitope portion enables the immunogenic fusion polypeptide to break tolerance in order for antibodies to be made that react with endogenous BLSA. By “breaking tolerance” is meant forcing an organism to mount an immune response to a protein, such as endogenous BLSA, that the organism does not normally find immunogenic.

DNA vaccines recently have been shown to be a promising approach for immunization against a variety of infectious diseases. Michel, M L et al., Huygen, K, et al., and Wang, B, et al. Delivery of naked DNAs containing microbial antigen genes can induce antigen-specific immune responses in the host. The induction of antigen-specific immune responses using DNA-based vaccines has shown some promising effects. Wolff, J. A., et al. Recent studies have demonstrated the potential feasibility of immunization using a DNA-mediated vaccine for CEA and MUC-1. Conry, R. M., et al. and Graham, R. A., et al.

DNA-based vaccination has been shown to have a greater degree of control of antigen expression, toxicity and pathogenicity over live attenuated virus immunization. The construction, operation and use of the above pharmaceutically acceptable carriers for DNA vaccination and the above delivery vehicles are described in detail in U.S. Pat. No. 5,705,151 to Dow et al., entitled “gene therapy for T cell regulation”, which is directed at anti-cancer treatment, and is hereby incorporated by reference as if fully set forth herein.

In another aspect, the present invention provides a method for immunizing a patient against B-cell lymphoma or other B-cell mediated diseases comprising injecting BLSA or an immunogenic fragment thereof into the patient. The BLSA or immunogenic fragment can be injected alone or in combination with suitable adjuvants and/or other antigens, as well as other therapeutics.

Generally, antigens are presented to the immune system using major histocompatibility complex (MHC) molecules, i.e., MHC Class I molecules and MHC Class II molecules. Endogenous or self antigens, such as tumor antigens like BLSA, are usually bound to MHC class I molecules and presented to cytotoxic T cells (“CTL”). Exogenous antigens, such as viral antigens, are usually bound to MHC Class II molecules and presented to T cells that interact with B cells to produce antibodies.

Antigens presented via the Class II pathway, known as MHC Class II-restricted antigens or Class II antigens, are recognized by and activate T cells. These activated T cells cause a complete immune response to the Class II antigens. Because self antigens normally are not presented to the immune system through the MHC Class II pathway, the immune system does not recognize these self antigens as foreign and does not form a complete immune response to such antigens.

In one embodiment of the present invention, the BLSA antigen is injected in combination, simultaneously or contemporaneously, with other antigens that are designed to stimulate or manipulate the immune response. Preferably, the BLSA antigen is injected as part of a construct comprising the BLSA antigen and other antigens that are designed to induce a cellular immune response. Such other antigens are designed to enhance antigen presentation to T cells and induce a more potent immune response to antigens such as BLSA that typically elicit an incomplete immune response because they are not recognized by the immune system as foreign antigens.

Typically, BLSA is injected in combination with Class II antigens. Use of other antigens to stimulate the immune system via the MHC Class II pathway in combination with the BLSA antigen, which may be recognized by the immune system as a self antigen that elicits a weak or incomplete immune response, helps to ensure that the BLSA antigen will be treated by the immune system as a foreign antigen that elicits a complete immune system response. Preferably, the BLSA antigen and the Class II antigen are part of a construct wherein the antigens are part of a single molecule. In another aspect, the present invention provides a construct comprising a BLSA antigen and another antigen in a single molecule. Preferably, the other antigen is a Class II antigen.

### Expression Vectors

Recombinant expression vectors containing a nucleotide sequence encoding the polypeptide can be prepared using well known techniques. The expression vectors include a nucleotide sequence operably linked to suitable transcriptional or translational regulatory nucleotide sequences such as those derived from mammalian, microbial, viral, or insect genes. Examples of regulatory sequences include transcriptional promoters, operators, enhancers, mRNA ribosomal binding sites, and appropriate sequences which control transcription and translation initiation and termination. Nucleotide sequences are “operably linked” when the regulatory sequence functionally relates to the nucleotide sequence for the appropriate polypeptide. Thus, a promoter nucleotide sequence is operably linked to a BLSA sequence if the promoter nucleotide sequence controls the transcription of the appropriate nucleotide sequence.

The ability to replicate in the desired host cells, usually conferred by an origin of replication and a selection gene by which transformants are identified, may additionally be incorporated into the expression vector.

In addition, sequences encoding appropriate signal peptides that are not naturally associated with BLSA can be incorporated into expression vectors. For example, a nucleotide sequence for a signal peptide (secretory leader) may be fused in-frame to the polypeptide sequence so that the polypeptide is initially translated as a fusion protein comprising the signal peptide. A signal peptide that is functional in the intended host cells enhances extracellular secretion of the appropriate polypeptide. The signal peptide may be cleaved from the polypeptide upon secretion of polypeptide from the cell.

### Host Cells

Suitable host cells for expression of BLSA include prokaryotes, yeast, archae, and other eukaryotic cells. Appropriate cloning and expression vectors for use with bacterial, fungal, yeast, and mammalian cellular hosts are well known in the art, e.g., Pouwels et al. Cloning Vectors: A Laboratory Manual, Elsevier, New York (1985). The vector may be a plasmid vector, a single or double-stranded phage vector, or a single or double-stranded RNA or DNA viral vector. Such vectors may be introduced into cells as polynucleotides, preferably DNA, by well known techniques for introducing DNA and RNA into cells. The vectors, in the case of phage and viral vectors also may be and preferably are introduced into cells as packaged or encapsulated virus by well known techniques for infection and transduction. Viral vectors may be replication competent or replication defective. In the latter case viral propagation generally will occur only in complementing host cells. Cell-free translation systems could also be employed to produce the protein using RNAs derived from the present DNA constructs.

Prokaryotes useful as host cells in the present invention include gram negative or gram positive organisms such as E. coli or Bacilli. In a prokaryotic host cell, a polypeptide may include a N-terminal methionine residue to facilitate expression of the recombinant polypeptide in the prokaryotic host cell. The N-terminal Met may be cleaved from the expressed recombinant BLSA polypeptide. Promoter sequences commonly used for recombinant prokaryotic host cell expression vectors include β-lactamase and the lactose promoter system.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct an expression vector using pBR322, an appropriate promoter and a DNA sequence are inserted into the pBR322 vector. Other commercially available vectors include, for example, pKK223-3 (Pharmacia Fine Chemicals, Uppsala, Sweden), pGEM1 (Promega Biotec, Madison, Wis., USA), and the pET (Novagen, Madison, Wis., USA) and pRSET (Invitrogen Corporation, Carlsbad, Calif., USA) series of vectors (Studier, F. W., J. Mol. Biol. 219: 37 (1991); Schoepfer, R. Gene 124: 83 (1993)).

Promoter sequences commonly used for recombinant prokaryotic host cell expression vectors include T7, (Rosenberg, A.H., Lade, B. N., Chui, D-S., Lin, S-W., Dunn, J. J., and Studier, F. W. (1987) Gene (Amst.) 56, 125-135), β-lactamase (penicillinase), lactose promoter system (Chang et al., Nature 275:615, (1978); and Goeddel et al., Nature 281:544, (1979)), tryptophan (trp) promoter system (Goeddel et al., Nucl. Acids Res. 8:4057, (1980)), and tac promoter (Maniatis, Molecular Cloning: A Laboratory Manual, Cold Spring Harbor Laboratory, p. 412 (1982)).

Yeasts useful as host cells in the present invention include those from the genus Saccharomyces, Pichia, K. Actinomycetes and Kluyveromyces. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, glucose-6-phosphate isomerase, 3phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991). Other suitable promoters and vectors for yeast and yeast transformation protocols are well known in the art.

Yeast transformation protocols are known to those of skill in the art. One such protocol is described by Hinnen et al., Proceedings of the National Academy of Sciences USA, 75:1929 (1978). The Hinnen protocol selects for Trp.sup.+ transformants in a selective medium, wherein the selective medium consists of 0.67% yeast nitrogen base, 0.5% casamino acids, 2% glucose, 10 μg/ml adenine, and 20 μg/ml uracil.

Mammalian or insect host cell culture systems well known in the art could also be employed to express recombinant BLSA, e.g., Baculovirus systems for production of heterologous proteins in insect cells (Luckow and Summers, Bio/Technology 6:47 (1988)) or Chinese hamster ovary (CHO) cells for mammalian expression may be used. Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art.

BLSA may, when beneficial, be expressed as a fusion protein that has the BLSA attached to a fusion segment. The fusion segment often aids in protein purification, e.g., by permitting the fusion protein to be isolated and purified by affinity chromatography. Fusion proteins can be produced by culturing a recombinant cell transformed with a fusion nucleic acid sequence that encodes a protein including the fusion segment attached to either the carboxyl and/or amino terminal end of the protein. Preferred fusion segments include, but are not limited to, glutathione-S-transferase, β-galactosidase, a poly-histidine segment capable of binding to a divalent metal ion, and maltose binding protein.

### Expression and Recovery

According to the present invention, isolated and purified BLSA may be produced by the recombinant expression systems described above. The method comprises culturing a host cell transformed with an expression vector comprising a nucleotide sequence that encodes the polypeptide under conditions sufficient to promote expression of the polypeptide. The polypeptide is then recovered from culture medium or cell extracts, depending upon the expression system employed. As is known to the skilled artisan, procedures for purifying a recombinant polypeptide will vary according to such factors as the type of host cells employed and whether or not the recombinant polypeptide is secreted into the culture medium. When expression systems that secrete the recombinant polypeptide are employed, the culture medium first may be concentrated. Following the concentration step, the concentrate can be applied to a purification matrix such as a gel filtration medium. Alternatively, an anion exchange resin can be employed, e.g., a matrix or substrate having pendant diethylaminoethyl (DEAE) groups. The matrices can be acrylamide, agarose, dextran, cellulose, or other types commonly employed in protein purification. Also, a cation exchange step can be employed. Suitable cation exchangers include various insoluble matrices comprising sulfopropyl or carboxymethyl groups. Further, one or more reversed-stage high performance liquid chromatography (RP-HPLC) steps employing hydrophobic RP-HPLC media (e.g., silica gel having pendant methyl or other aliphatic groups), ion exchange-HPLC (e.g., silica gel having pendant DEAE or sulfopropyl (SP) groups), or hydrophobic interaction-HPLC (e.g., silica gel having pendant phenyl, butyl, or other hydrophobic groups) can be employed to further purify the protein. Some or all of the foregoing purification steps, in various combinations, are well known in the art and can be employed to provide an isolated and purified recombinant polypeptide.

Recombinant polypeptide produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification, or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

### Agonists and Antagonists Screening

In another aspect, the present invention provides a screening method for identifying BLSA agonists and antagonists. The screening method comprises exposing a BLSA to a potential BLSA agonist/BLSA antagonist and determining whether the potential agonist/antagonist binds to BLSA. If the potential agonist/antagonist binds, there is a strong presumption that the potential agonist/antagonist will actually function as an agonist or antagonist when administered in vivo to a patient and exposed to the native BLSA. The BLSA agonists and BLSA antagonists identified using the method can be characterized as an agonist or an antagonist by exposing cells capable of producing cytokines to the agonist/antagonist and measuring cytokine production in comparison to non-exposed cells. Agonists will increase cytokine production; antagonists will decrease cytokine production. Another method for screening comprises transfecting the cells with a reporter gene constructs that contains BLSA DNA binding sequences. Preferably, the potential agonist/antagonist is an organic compound or polypeptide, including antibodies. The screening methods are useful for identifying compounds that may function as drugs for preventing or treating diseases, particularly diseases characterized by relatively low or relatively high cytokine production compared to non-disease states.

### BLSA Expression Modulation

In yet another aspect, the present invention provides a method for blocking or modulating the expression of a cellular BLSA by interfering with the transcription or translation of a DNA or RNA polynucleotide encoding the BLSA. The method comprises exposing a cell capable of expressing a BLSA to a molecule that interferes with the proper transcription or translation of a DNA or RNA polynucleotide encoding the BLSA. The molecule can be an organic molecule, a bioorganic molecule, an antisense nucleotide, a RNAi nucleotide, or a ribozyme.

In a preferred embodiment, the method comprises blocking or modulating the expression of cellular BLSA by exposing a cell to a polynucleotide that is antisense to or forms a triple helix with BLSA DNA or with DNA regulating expression of BLSA. The cell is exposed to antisense polynucleotide or triple helix-forming polynucleotide in an amount sufficient to inhibit or regulate expression of the BLSA activating receptor. Also, the present invention provides a method for blocking or modulating expression of BLSA in an animal by administerng to the animal a polynucleotide that is antisense to or forms a triple helix with BLSA encoding DNA or with DNA regulating expression of BLSA-encoding DNA. The animal is administered antisense polynucleotide or triple helix-forming polynucleotide in an amount sufficient to inhibit or regulate expression of BLSA in the animal. Preferably, the antisense polynucleotide or triple helix-forming polynucleotide is a DNA or RNA polynucleotide.

Methods for exposing cells to antisense polynucleotides and for administering antisense polynucleotides to animals are well known in the art. In a preferred method, the polynucleotide is incorporated into the cellular genome using know methods and allowed to be expressed inside the cell. The expressed antisense polynucleotide binds to polynucleotides coding for BLSA and interferes with their transcription or translation.

### Disease Predisposition Diagnostic

In another aspect, the present invention provides a method for diagnosing the predisposition of a patient to develop diseases caused by the unregulated expression of BLSA. The invention is based upon the discovery that the presence of or increased amount of BLSA in certain patient cells, tissues, or body fluids indicates that the patient is predisposed to certain immune diseases. In one embodiment, the method comprises collecting a cell, tissue, or body fluid sample known to contain b-CELLS from a patient, analyzing the tissue or body fluid for the level of BLSA in the tissue, and predicting the predisposition of the patient to certain immune diseases based upon the level of BLSA detected in the tissue or body fluid. In another embodiment, the method comprises collecting a cell, tissue, or body fluid sample known to contain a defined level of BLSA from a patient, analyzing the tissue or body fluid for the amount of BLSA in the tissue, and predicting the predisposition of the patient to certain immune diseases based upon the change in the amount of BLSA in the tissue or body fluid compared to a defined or tested level extablished for normal cell, tissue, or bodly fluid. The defined level of BLSA may be a known amount based upon literature values or may be determined in advance by measuring the amount in normal cell, tissue, or body fluids. Specifically, determination of BLSA levels in certain tissues or body fluids permits specific and early, preferably before disease occurs, detection of immune diseases in the patient. Immune diseases that can be diagnosed using the present method include, but are not limited to, the immune diseases described herein. In the preferred embodiment, the tissue or body fluid is peripheral blood, peripheral blood leukocytes, biopsy tissues such as lung or skin biopsies, and synovial fluid and tissue.

### Disease Prevention and Treatment

The dosages of BLSA agonist or antagonist vary according to the age, size, and character of the particular mammal and the disease. Skilled artisans can determine the dosages based upon these factors. The agonist or antagonist can be administered in treatment regimes consistent with the disease, e.g., a single or a few doses over a few days to ameliorate a disease state or periodic doses over an extended time to prevent allergy or asthma.

The agonists and antagonists can be administered to the mammal in any acceptable manner including by injection, using an implant, and the like. Injections and implants are preferred because they permit precise control of the timing and dosage levels used for administration. The agonists and antagonists are preferably administered parenterally. As used herein parenteral administration means by intravenous, intramuscular, or intraperitoneal injection, or by subcutaneous implant.

When administered by injection, the agonists and antagonists can be administered to the mammal in a injectable formulation containing any biocompatible and agonists and antagonists compatible carrier such as various vehicles, adjuvants, additives, and diluents. Aqueous vehicles such as water having no nonvolatile pyrogens, sterile water, and bacteriostatic water are also suitable to form injectable solutions. In addition to these forms of water, several other aqueous vehicles can be used. These include isotonic injection compositions that can be sterilized such as sodium chloride, Ringer's, dextrose, dextrose and sodium chloride, and lactated Ringer's. Nonaqueous vehicles such as cottonseed oil, sesame oil, or peanut oil and esters such as isopropyl myristate may also be used as solvent systems for the compositions. Additionally, various additives which enhance the stability, sterility, and isotonicity of the composition including antimicrobial preservatives, antioxidants, chelating agents, and buffers can be added. Any vehicle, diluent, or additive used would, however, have to be biocompatible and compatible with the agonists and antagonists according to the present invention.

### BLSA Polypeptide Diagnostic

The antibodies of the present invention may also be used in a diagnostic method for detecting BLSA expressed in specific cells, tissues, or body fluids or their components. The method comprises exposing cells, tissues, or body fluids or their components to an antibody of the present invention and determining if the cells, tissues, or body fluids or their components bind to the antibody. Cells, tissues, or body fluids or their components that bind to the antibody are diagnosed as cells, tissues, or body fluids that contain BLSA. Such method is useful for determining if a particular cell, tissue, or body fluid is one of a certain type of cell, tissue, or body fluid previously known to contain BLSA. Various diagnostic methods known in the art may be used, e.g., competitive binding assays, direct or indirect sandwich assays, and immunoprecipitation assays conducted in either heterogeneous or homogeneous stages.

### Knockout Animals

In another aspect, the present invention provides a knockout animal comprising a genome having a heterozygous or homozygous disruption in its endogenous BLSA gene that suppresses or prevents the expression of biologically functional BLSA proteins. Preferably, the knockout animal of the present invention has a homozygous disruption in its endogenous BLSA gene. Preferably, the knockout animal of the present invention is a mouse. The knockout animal can be made easily using techniques known to skilled artisans. Gene disruption can be accomplished in several ways including introduction of a stop codon into any part of the polypeptide coding sequence that results in a biologically inactive polypeptide, introduction of a mutation into a promoter or other regulatory sequence that suppresses or prevents polypeptide expression, insertion of an exogenous sequence into the gene that inactivates the gene, and deletion of sequences from the gene.

Several techniques are available to introduce specific DNA sequences into the mammalian germ line and to achieve stable transmission of these sequences (transgenes) to each subsequent generation. The most commonly used technique is direct microinjection of DNA into the pronuclcus of fertilized oocytes. Mice or other animals derived from these oocytes will be, at a frequency of about 10 to 20%, the transgenic founders that through breeding will give rise to the different transgenic mouse lines. Methods for generating transgenic animals via embryo manipulation and microinjection, particularly animals such as mice, have become conventional in the art, e.g., U.S. Pat. Nos. 4,736,866, 4,870,009, and 4,873,191 and in Hogan, B., Manipulating the Mouse Embryo, (Cold Spring Harbor Laboratory Press, Cold Spring Harbor, N.Y., 1986). Similar methods are used for production of other transgenic animals.

Embryonic stem cell (“ES cell”) technology can be used to create knockout mice (and other animals) with specifically deleted genes. Totipotent embryonic stem cells, which can be cultured in vitro and genetically modified, are aggregated with or microinjected into mouse embryos to produce a chimeric mouse that can transmit this genetic modification to its offspring. Through directed breeding, a mouse can thus be obtained that lacks this gene. Several other methods are available for the production of genetically modified animals, e.g., the intracytoplasmic sperm injection technique (ICSI) can be used for transgenic mouse production. This method requires microinjecting the head of a spermatocyte into the cytoplasm of an unfertilized oocyte, provoking fertilization of the oocyte, and subsequent activation of the appropriate cellular divisions of a preimplantation embryo. The mouse embryos thus obtained are transferred to a pseudopregnant receptor female. The female will give birth to a litter of mice. In ICSI applied to transgenic mouse production, a sperm or spermatocyte heads suspension is incubated with a solution containing the desired DNA molecules (transgene). These interact with the sperm that, once microinjected, act as a carrier vehicle for the foreign DNA. Once inside the oocyte, the DNA is integrated into the genome, giving rise to a transgenic mouse. This method renders higher yields (above 80%) of transgenic mice than those obtained to date using traditional pronuclear microinjection protocols.

### Gene Therapy

Since BLSA is highly expressed in several human abnormal B-type leukemia cell lines. BLSA can be used as a target in the area of gene therapy for various types of B-cell leukemia (e.g.Burkitts's lymphoma and immunoblastic B cell lymphoma etc). Gene therapy can either be applied ex vivo or in vivo, and BLSA can be targeted at the levels of DNA, RNA or its protein product. For example, BLSA specific oligodeoxynucleotides can be used to form a triple helix with purine rich double stranded DNA sequence to inactivate BLSA gene inside the cancer cell. At the RNA level, one can use antisense techniques to prevent transport and translation of the BLSA by providing a complementary RNA molecule (e.g., Collins, J., Herman, P., Schuch, C. and Babgy G. (1992)) c-myc antisense oligonucleotides inhibit the colony-forming capacity of Colo 320 colonic carcinoma cells. Journal of Clinical Investigation 89:1523-1527; Ebbinghouse, S., Gee, I., Rodu, B., Mayfield, C. and Miller, D. (1993) Triplex formation inhibits HER2/neu transcription in vitro. Journal of Clinical Investigation 92:2433-2439.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

### Identification of BLSA

BLSA was identified by searching human EST data using the Hidden Markov Model (HMM) of the immunoglobulin (Ig) domain. The HMM was originally built from an alignment of 113 confirmed Ig domains and calibrated using the program HMMER (S.R. Eddy. Profile hidden Markov models. Bioinformatics 14:755-763, 1998). The HMM was obtained from the Pfam (version 6,6, http://pfam.wustl.edu/) database and used to search the human EST data. To reduce the Ig HMM search time, we generated a total of 189,623 EST contigs/consensus sequences from 2.9 million public EST sequences, which were stored and organized using a relational database system.

The search was performed using a unique software system, which allowed automation of the process. Briefly, a fasta formatted file containing all the Human EST contigs was generated, which was then searched by the program estwisedb (http://www.sanger.ac.uk/Software/Wise2) for matches with the Ig HMM. The results were processed and evaluated, and thresholds of raw score of estimated E-value were selected to minimize both false negative and false positive rates. 555 EST contigs were selected for further analysis. All 555 EST contigs were blasted against the non-redundant protein database accumulated from all species. The resulting hits were screened for interesting candidates based on the novelty of the sequences and the sequence similarity to Ig domain. For each Ig domain containing candidate, a series of in silico characterizations were carried out, which included analysis of the location in the genome and its relationship to its adjacent sequences, UniGene cluster annotation, coding region identification and verification, evidence from multiple sources that the domain is or includes an EST and that it is expressed in different tissues and cell lines. A total of 10 candidates were selected for further experimental characterization.

### Example 2

### Molecular Cloning and Characterization of BLSA

The predicted coding region of BLSA were cloned into pCR3.1-Topo vector (Invitrogen) in frame with the 3′ V5 and His tag sequences by PCR using Daudi cell line cDNA as templates. This cDNA was then transiently transfected into 293T cells with Lipofectamine 2000 for expression. The whole cell protein sample was prepared by re-suspending 3×10⁵ cells in 100 μl of ddH₂O, and heated at 98° C. for 5 minutes after adding equal volume of 2× sample loading buffer. The proteins were separated in a 15% SDS-PAGE and transferred to membrane. The tagged BLSA protein is detected as a 50 kD protein band by Western blot with anti-V5 mAb. This protein band was not present in the cells transfected with plasmid vector-only

### Example 3

Quantitative Real-Time PCR Analysis of BLSA mRNA Expression

Two sets of oligonucleotide primers:

| | |
|---|---|
| (5′-GTGAACCCTTCCACCTGATTGT and | (SEQ ID NO 21) |
| 5′-GACCTTGGAGGATCAGCCAGT; | (SEQ ID NO 22 |
| 5′-CGGGCCTAACAGGGAATTCT and | (SEQ ID NO 23) |
| 5′-CCCGCTGTCTGCCTTTTGTA | (SEQ ID NO 24)) |

were selected from the BLSA nucleotide sequences using Primer Express 2.0 (Applied Biosystems, Inc.) and were synthesized and used in real-time PCR reactions to measure the expression of BLSA. RNAs were isolated in order to measure the level of expression of BLSA in the following cells: Daudi, a Burkitt's lymphoma cell line; Ramos, a B lymphocyte Burkitt's lymphoma; Raji, a B lymphocyte Burkitt's lymphoma cell line; SKO-007, a mycloma cell line; Clone 15 of HL-60, an acute promyelocytic leukemia cell line; JMI, a pre-B lymphoblast lymphoma cell line; REH, a pro-B acute lymphocytic leukemia cell line; THP-1, acute monocytic leukemia; HMC-1; an immature human mast cell line; HUVEC; primary human vascular endothelial cells; primary B-cells; CD34+ progenitor cells; primary basophils; neutrophils; monocytes; and HPB-ALL, a T cell leukemia cell line.

Real-time quantitative PCR (Taqman) was performed with the ABI Prism 7900 (Applied Biosystems, Inc.) sequence detection system according to the manufacture's instructions. Equal amounts of each of the RNAs from the cell lines indicated above were used as PCR templates in reactions to obtain the threshold cycle (Cₜ), and the Cₜ was normalized using the known Cₜ from 18S RNAs to obtain ΔCₜ. To compare relative levels of gene expression of BLSA in different cell lines, ΔΔCₜ values were calculated by using the lowest expression level as the base, which were then converted to real fold expression difference values.

BLSA mRNA was found to be highly expressed in B-cell lymphoma cell lines, Daudi, Ramos and Raji, and in pre-B lymphoma cells, designated JMI. Very low level expression was found in the proB cells REH and in primary B cells. The level of expression in HPB-ALL, THP-1, peripheral lymphocytes, monocytes, human endothelial cells, CD34+ progenitor cells, mast cells, basophils, and neutrophils was negligible (See Tables 1 and 2).

TABLE 1
Relative expression (real fold difference) of BLSA in cell line set I

| | Relative Expression |
|---|---|
| Cells | (arbitrary unit) |
| Daudi cells | 75480.5 |
| Monocytes | 380.0 |
| HMC-1 | 13.4 |
| B-cell | 1573.7 |
| Basophils | 184.6 |
| Mast cells (week 1) | 34.8 |
| Mast cells (week 5) | 206.8 |
| Mast cells (week 9) | 788.0 |
| Mast cells (week 9, IgE) | 634.1 |
| HPB-ALL | 17.9 |
| Lymphocytes | 135.6 |
| Neutrophils | 118.4 |
| HUVEC | 1.0 |

TABLE 2
Relative expression (real fold difference) of BLSA in cell line set II

| | Relative Expression |
|---|---|
| Cells | (arbitrary unit) |
| Daudi cells | 95840.7 |
| Ramos (RA 1) | 24042.3 |
| Raji | 79674.2 |
| SKO-007 | 16.5 |
| Clone 15 HL-60 | 1.0 |
| JM1 | 122395.4 |
| Reh | 392.8 |
| Mast cells (week 1) | 29.1 |
| Mast cells (week 5) | 173.0 |
| Mast cells (week 9) | 47.2 |
| Mast cells (week 9, IgE) | 71.2 |
| PRIMARY B(50%) | 160.8 |
| HMC-1 | 18.8 |
| THP-1 | 4.3 |
| HUVEC | 1.6 |

### Example 4

### Anti-BLSA Monoclonal Antibody Generation

Anti-BLSA monoclonal antibodies were generated by immunizing mice with plasmid encoding BLSA using a Gene Gun. Individual anti BLSA monoclonal antibody was characterized by ELISA and Western blot using recombinant BLSA protein.

### Example 5

### Expression of BLSA Protein in B Cell Lymphoma Cell Lines

To determine whether BLSA protein is expressed in B cell lymphoma cell lines, we performed immunofluorescence experiments. Briefly, 25,000 cells were cytospinned onto glass slides and air-dried. Cells were fixed with Carnoy's Fix (60% ethanol, 30% chloroform and 10% acetic acid) for 10 minutes at room temperature, and washed with PBS three times. Cells were pre-blocked with block solution (1% horse serum, 1% TRITON X-100, 2% rabbit serum, 1% BSA, and 1% goat serum in PBS) on ice for 30 minutes and incubated with anti-BLSA mAb (1 ug/ml in 1% BSA in PBS) for 30 minutes at room temperature. Cells were then washed three times and incubated with goat anti-mouse IgG (H+L)-FITC (Jackson Immuno Lab) at 1:100 dilution for 30 minutes at room temperature. Cells were washed, air dried and covered with coverslides. Fluorescence staining was examined using a fluorescence microscope and results recorded using Snap-Shot software. It was found that BLSA was detected in B cell lymphoma Daudi, human pre-B lymphoblast CRL10423 and 1596, but not in a T cell line Jurkat or the mast cell line HMC-1 (Table 3).

TABLE 3
Immunofluorescence staining

| Cell | | |
|---|---|---|
| line name | Cell line description | Anti-BLSA staining results |
| HMC-1 | immature human Mast cell line | Negative |
| Jurkat | human T leukemic cell line | Negative |
| CRL | human pre-B lymphoblast | Positive |
| 10423 | | |
| CRL1596 | human Burkitt lymphoma (EBV | Positive |
| | negative) derived cell line | |
| Daudi | human lymphoma-derived B cell | Positive |
| | line | |

What is claimed:

1. A molecule that binds to B-cell Specific Antigen (BLSA) (SEQ ID NO 2).

2. The molecule of claim 1, wherein the molecule is an agonist.

3. The molecule of claim 1, wherein the molecule is an antagonist.

4. The molecule of any one of claims 1 to 3, wherein the molecule is an antibody, a peptide, a ligand, a small molecule, or an oligonucleotide.

5. The molecule of claim 4, wherein the molecule is an antibody or a binding fragment thereof.

6. The antibody of claim 5, wherein said antibody is monoclonal, chimeric, human, humanized, bispecific, or a heteroconjugate.

7. The antibody fragment of claim 5, wherein the fragment is F(ab′)₂, F(ab)₂, Fab′, Fab.

8. A composition comprising the molecule of any one of claims 1 to 7 and a physiologically acceptable carrier, diluent, excipient, and/or additive.

9. A method for treating a B-cell mediated disease comprising administering the composition of claim 8.

10. The method of claim 9, wherein the B-cell mediated disease is B-cell lymphoma.

11. The method of claim 9, wherein the B-cell mediated disease is selected from the group consisting of follicular cell lymphomas, diffuse small lymphocytic lymphoma/chronic lymphocytic leukemia (CLL), lymphoplasmacytoid/Waldenstrom's Macroglobulinemia, Marginal zone lymphoma and Hairy cell leukemia.

12. The method of claim 9, wherein the B-cell mediated disease is selected from the group consisting of diffuse large cell lymphoma, Burkitt's lymphoma/diffuse small non-cleaved cell lymphoma, Lymphoblastic lymphoma, Mantle cell lymphoma and AIDS-related lymphoma.

13. A vaccine for the treatment of a B-cell lymphoma comprising a polypeptide comprising an amino sequence selected from the group consisting of: SEQ ID NO:2; a variant of SEQ ID NO:2; and a fragment of SEQ ID NO:2, wherein the peptide contains at least one epitope.

14. A vaccine for the treatment of B-cell lymphoma comprising a polypeptide encoded by an isolated polynucleotide comprising a nucleotide sequence selected from the group consisting of: SEQ ID NO:1; a variant of SEQ ID NO:1; and a fragment of SEQ ID NO:1, wherein the peptide contains at least one epitope.

15. A method for immunizing a patient against B-cell lymphoma or other B-cell mediated disease, comprising administering the vaccine of claim 13 or claim 14.

16. The method of claim 15, further comprising administering the vaccine to a patient in combination with an adjuvant, simultaneously or consecutively.

17. The method of claim 15, further comprising administering the vaccine to a patient in combination with a second antigen, simultaneously or consecutively.

18. The method of claim 17, wherein the second antigen is a Class II antigen.

19. A DNA construct comprising a nucleic acid sequence which expresses BLSA or an immunogenic fragment thereof, operably linked to a promoter.

20. The construct of claim 19, further comprising a class II antigen.

21. An isolated antibody produced in response to the method of claim 15.

22. A method for inducing an immune response in a mammal against BLSA comprising administering a composition comprising a DNA molecule that encodes BLSA, said DNA molecule operatively linked to regulatory sequences which control the expression of said DNA molecule, wherein BLSA is expressed in said cells and an immune response is generated against BLSA.

23. A BLSA peptide comprising at least one epitope that induces a cytotoxic T-Lymphocyte (CTL) response.

24. A method of inducing an immune response in a mammal against BLSA comprising administering a cytotoxic T-Lymphocyte (CTL)-inducing peptide.

25. A method of inducing an immune response in a mammal against BLSA comprising administering a vector that expresses a cytotoxic T-Lymphocyte (CTL)-inducing peptide of BLSA.

26. A host cell comprising a vector that expresses a cytotoxic T-Lymphocyte (CTL)-inducing peptide of BLSA.

27. A method for inhibiting the expression of BLSA by administering a composition that decreases the rate of translation of BLSA in a B-cell lymphoma cell, comprising exposing the cell to an antisense nucleic acid or antisense nucleic acid mimic that hybridizes to said RNA species or to DNA encoding said RNA species.

28. A method for screening for an agent with the ability to modulate expression of BLSA comprising the steps of:
(a) contacting a cell comprising the gene encoding BLSA with a candidate agent under conditions sufficient to permit modulation of the level of mRNA transcribed from the BLSA gene;
(b) isolating mRNA;
(c) comparing the amount of detected mRNA with an amount detected in the absence of candidate agent, and therefrom determining the ability of the candidate agent to modulate expression of BLSA.

29. A method for screening for an agent with the ability to bind BLSA comprising the steps of:
(a) contacting BLSA with a candidate agent under conditions sufficient to permit binding;
(b) detecting the presence of a BLSA/agent complex.

30. The method according to claim 28 or 29, wherein the candidate agent is present within a small molecule combinatorial library.

31. A method for blocking or modulating the expression of a cellular BLSA by interfering with the transcription or translation of a DNA or RNA polynucleotide encoding the BLSA comprising exposing a cell capable of expressing a BLSA to a molecule that interferes with the transcription or translation of a DNA or RNA polynucleotide encoding the BLSA.

32. The method of claim 31, wherein the molecule is an antisense molecule, an RNAi molecule, or a ribozyme that interferes with the proper transcription or translation of a DNA or RNA polynucleotide encoding the BLSA.

33. The method of claim 32, wherein the molecule is an antisense nucleotide that interferes with the proper transcription or translation of a DNA or RNA polynucleotide encoding the BLSA.

34. A method for diagnosing the predisposition of a patient to develop a B-cell mediated disease caused by the unregulated expression of BLSA, comprising:
collecting a cell, tissue, or body fluid sample from a patient;
analyzing the tissue or body fluid for the presence of BLSA; and
predicting the predisposition of the patient to B-cell mediated diseases based upon the level of expression of BLSA in the tissue or body fluid.

35. A method for diagnosing the predisposition of a patient to develop a B-cell mediated disease caused by the unregulated expression of BLSA, comprising:
collecting a cell, tissue, or body fluid sample known to contain a defined level of BLSA from a patient;
analyzing the tissue or body fluid for the amount of BLSA in the tissue; and
predicting the predisposition of the patient to certain immune diseases based upon the change in the amount of BLSA in the tissue or body fluid compared to a defined or tested level extablished for normal cell, tissue, or bodly fluids.

35. A method for preventing or treating BLSA protein mediated diseases in a mammal comprising administering a disease preventing or treating amount of a BLSA agonist or antagonist to the mammal.

36. The method of claim 19 wherein the BLSA agonist or antagonist is an antibody.

37. A method for producing an antibody that binds to BLSA, comprising a method selected from the group consisting of:
using isolated BLSA or antigenic fragments thereof as an antigen;
using host cells that express recombinant BLSA as an antigen; and
using DNA expression vectors containing the BLSA gene to express BLSA as an antigen for producing antibodies.

38. The antibody produced using the method of claim 37.

39. The antibody of claim 38 selected from the group consisting of polyclonal, monoclonal, humanized, human, bispecific, and heteroconjugate antibodies.

40. A diagnostic method for detecting BLSA expressed in specific cells, tissues, or body fluids, comprising:
exposing cells, tissues, or body fluids or their components to the antibodies of claim 38; and
determining if the cells, tissues, or body fluids or their components bind to the antibody.

41. A method for isolating and purifying BLSA from recombinant cell culture, contaminants, and native environments, comprising:
exposing a composition containing BLSA and contaminants to an antibody capable of binding to BLSA;
allowing the BLSA to bind to the antibody;
separating the antibody-BLSA complexes from the contaminants; and
recovering the BLSA from the complexes.

42. The method of claim 25 wherein the antibody is an antibody of claim 38.

43. A transgenic knockout animal whose genome comprises a heterozygous or homozygous disruption in its endogenous BLSA gene that suppresses or prevents the expression of biologically functional BLSA proteins.

44. A method for imaging lesions characteristic of certain lymphomas, comprising the steps of:
obtaining monoclonal antibody specific to BLSA; labeling said antibody; contacting said labeled antibody with a biological sample obtained from a mammal; and imaging said label.

45. A method for detecting the level of BLSA in a cell comprising performing quantitative PCR.

46. A method for detecting BLSA in a cell comprising performing immunofluorescence staining.
